(19) United States
(12) Patent Application Publication
(10) Pub. No.: US 2018/0223243 A1
(43) Pub. Date: Aug. 9, 2018

(54) METHOD AND SYSTEM FOR COMPENSATING PERTURBED MEASUREMENTS

(71) Applicant: Stratophase Ltd, Hampshire (GB)

(72) Inventors: Gregory Daniel Emmerson, Winchester, Hants (GB); Peter George Robin Smith, Romsey, Hants (GB)

(21) Appl. No.: 15/750,229

(22) PCT Filed: Aug. 2, 2016
(86) PCT No.: PCT/GB2016/052373

§ 371 Date: Feb. 5, 2018

(30) Foreign Application Priority Data
Aug. 4, 2015 (GB) 1513799.5
Feb. 2, 2016 (GB) 1601853.3

(51) Int. Cl. C12M 1/36; C12M 1/34

(52) U.S. Cl. CPC C12M 41/48; C12M 41/32; C12M 41/36

(57) ABSTRACT

A method for compensating perturbed measurements comprises: obtaining measurements with respect to time of a signal representing a parameter modified by a perturbing influence and a signal representing the perturbing influence and storing the measurements for each time in a data array as parameter data and perturbation data respectively; for each of the parameter data and the perturbation data: fitting a mathematical model to the data with respect to time; determining an error between the model and the data at each time; storing the errors in the data array as normalised parameter data and normalised perturbation data respectively; fitting a mathematical model to the normalised parameter data against the normalised perturbation data to obtain a correlation function representing correlation between the parameter data and the perturbation data; using the correlation function to calculate a correction factor representing the modification of the parameter data by the perturbing influence; using the correction factor to adjust the parameter data modification to obtained compensated parameter data; and outputting the compensated parameter data.

### BACKGROUND OF THE INVENTION

The present invention relates to a method and system for compensating measurements of a parameter of interest for the perturbing effects of a source of noise or interference.

There are many instances where a parameter which is measured or monitored during a process has its measurement accuracy comprised by some external factor that modifies the parameter, having the effect of introducing noise or noise-like perturbation or disruption to the measurement. Various techniques can be used to compensate for the modification, but if the effect of the external factor on the parameter is unknown or unpredictable, simple approaches such as look-up tables or formulaic corrections may not work well.

Consider a system performing a process from which a measurement is made. The measured signal responds to all or much process information, some of which is genuine information in that it represents a parameter of interest, and some of which is not of interest. This latter information has a spurious perturbing or disrupting effect on the genuine information, so as to mask the genuine information and introduce errors into measurement or monitoring of the parameter of interest. For simplicity in this application, the information which is not of interest will sometimes be referred to as “noise”, although it is generally not noise in the conventionally understood sense in the field of signal processing. In particular, the parameter of interest may have a dependence on the perturbing factor so that the parameter and the perturbing factor are not independent, but the behaviour of the parameter which is required is not its variation caused by the perturbation.

As an example, consider a measurement in which refractive index is the parameter of interest. This might be obtained using an optical sensor based on a Bragg grating in an optical waveguide, the sensor being structured so that the reflective and transmissive responses of the grating depend on the refractive index of a medium around the sensor. As is well-known, the refractive index of a medium varies with temperature. Additionally, the response of the grating will vary with the temperature of the sensor itself, as the waveguide expands or contracts. Thus, a varying environmental temperature will modify the refractive index measurement, and hence can be considered as a perturbing influence or source of noise. Typically, both measurements are also independently subject to drift from other factors that are not correlated.

A particular example of refractive index measurement which can be affected by temperature fluctuations lies in the field of bioreactors and chemical reactors, which are important in industry for the production of chemicals and bio-chemicals. A large class of these reactor processes operate in a mode where a reactor vessel is filled with one or more fluids containing chemical and biological entities which are then converted by biological or chemical reactions within the reactor, such as the fermentation of sugars to produce alcohols using a yeast organism as the biological entity. More complex processes can grow genetically modified mammalian cells or microbial cells, important in the production of pharmaceutical products. The refractive index of the reactor contents varies over the process as the contents evolves, so can yield information about the contents composition. It will also be modified by temperature changes, however. One may wish to remove the thermal information to obtain an accurate view of the underlying refractive index.

The growth conditions in a bioreactor (such as temperature, pH, oxygen concentration, amino-acid composition) must be controlled so as to maintain the health and productivity of the desirable organisms, often by mimicking the conditions found in vivo. Furthermore, the reaction vessel must be kept in an aseptic state to prevent unwanted bio-entities colonising the reactor. The bioreactor may operate for timescales ranging from hours to months, and during operation the conditions, including temperature, must be monitored and controlled to ensure the desired product. In many processes, controlled additions of nutrients (feeding events) are made to maintain the ongoing bio-process.

The control of simple properties such as feedback-based control of reactor temperature has been well-established for many decades. More recently, there is increased interest in providing feedback-based approaches to control the addition of nutrients. Such systems monitor a meaningful property of the reactor contents, make control decisions based on the property, and then act on the control decision to maintain the process by adding nutrients. One approach to monitoring is to identify a specific chemical in the reactor, for example by using off-line sampling of the reactor contents. An alternative proposed by the inventors is to use refractive index data (which is not specific to a particular chemical entity) to control nutrient addition for high quality automated reactor performance (GB 1416233.3). As mentioned, however, refractive index data which reflects the composition of the reactor contents are susceptible to temperature-induced “noise”.

Refractive index measurements may conveniently be made using a grating-based evanescent field optical sensor such as the Ranger Probe device produced by Stratophase Limited (www.stratophase.com), which offers significant advantages for real-time in-situ operation. In particular these sensors are able to operate in chemically hostile environments and are able to survive the high temperature steam-in-place operations routinely used in industry to sterilise bioreactor equipment. The sensors have a small physical size and thermal mass, and can be located directly within the fluid contents of the bioreactor. While this gives good refractive index monitoring, it also means that the sensor response follows temperature fluctuations within the fluid. It is common for fairly simple thermal management to be used in reactors to maintain a proper temperature range for product growth; temperature fluctuations in the order of fractions of a degree centigrade are common, and the temperature will also change when fluids such as nutrients are added. In many cases, and where reactions are run over weeks, the building temperature (diurnal temperature variation) will also affect the temperature of the fluid, and weekend effects are also seen during bioreactor runs exceeding a week. Thus it is desirable for the output of real-time refractive index sensors used for feeding event control to be compensated or corrected for temperature variability within the reacting fluid media.

Importantly, it should be noted that all fluids have a refractive index n that varies with temperature T (dn/dT); this should be taken into account in processing refractive index data measured from the fluid. In the present examples, refractive index is the parameter of interest and temperature is not of interest, but nevertheless causes a genuine change in the refractive index. One might consider absolute measurement of the temperature with a view to filtering its effect from the refractive index data, but such absolute measurements may not be available or convenient. Also, more subtle effects can occur. Temperature variation can cause stress within the refractive index sensor, resulting in small additional changes in the measured signal. Similarly, thermal lag or thermal variation across a sensor can also lead to slight signal variation. Note also that the composition of the fluid media in a bioreactor is changing over the timescale of the process due to production of the desired product, and thus the optical properties of the media (refractive index and optical scatter) will vary over time. This changing composition also alters the dn/dT of the fluid.

These factors all combine to make it very difficult to use a look-up or formulaic approach for temperature compensation. Also, the steam sterilisation requirement for sensors is an aggressive process, often involving reactive chemical species and potentially causing denaturation of proteins and resulting in baked-on residues of both organic and inorganic materials. This also makes a look-up based compensation approach difficult to achieve.

During actual bioreactor process runs a number of events will typically occur which not only alter the measured refractive index signal, but can also alter the relationship between the measurement channel for the refractive index and the measurement channel for temperature, including fouling from a build-up of organic material on the sensors; composition change, due both to addition of new matter and the production of product; spatial inhomogeneity in the composition of the media; variable state of the media due to clumping, aggregation, protein folding, precipitation and the like; stress change on the sensors, such as water diffusion that can affect both the glass and polymer encapsulant of the sensor; thermal flows if the sensor bridges external and in-reactor environments; and optical noise such as polarisation shift. These factors further complicate the relationship between the refractive index and temperature, again making a look-up or formulaic compensation problematic.

Consequently, there is a requirement for an improved technique to compensate a measured signal for interference, such as temperature-induced fluctuation in a measurement of refractive index.

### SUMMARY OF THE INVENTION

Accordingly, a first aspect of the present invention is directed to a method for compensating perturbed measurements, comprising: obtaining measurements with respect to time of a signal representing a parameter modified by a perturbing influence and a signal representing the perturbing influence (and possibly one or more other parameters) and storing the measurements for each time in a data array as parameter data and perturbation data respectively; for each of the parameter data and the perturbation data: fitting a mathematical model to the data with respect to time; determining an error between the model and the data at each time; storing the errors in the data array as normalised parameter data and normalised perturbation data respectively; fitting a mathematical model to the normalised parameter data against the normalised perturbation data to obtain a correlation function representing correlation between the parameter data and the perturbation data; using the correlation function to calculate a correction factor representing the modification of the parameter data by the perturbing influence; using the correction factor to adjust the parameter data for the modification to obtained compensated parameter data; and outputting the compensated parameter data.

The measured signal is compensated for the perturbation using correlation between the measured signal and a reference measurement which includes the perturbing information but lacks the genuine information. In the bioreactor example discussed above, reference measurement of the thermal information obtained in parallel with a signal measurement including the refractive index information and the thermal information can be used to compensate for temperature-induced modification by looking at correlation between the measurements, to produce improved or corrected data representing the refractive index.

The correction factor may be calculated by multiplying normalised perturbation data for a most recent time with a multiplier of a first degree of the correlation function mathematical model, with the compensated parameter data then being obtained by subtracting the correction factor from the parameter data for the most recent time.

The method may further comprise calculating one or more statistical coefficients of the correlation function that indicate the goodness of fit of the correlation function mathematical model; and using at least one of the statistical coefficients to scale a magnitude of the correction factor before using the correction factor to adjust the parameter data, such that the correction factor is reduced inversely with the goodness of fit, a better fit yielding a larger magnitude. In this way, the compensation of the measurement is most aggressive when the confidence in the correction is highest.

In an embodiment, the parameter data and the perturbation data are stored in the data array to create a full array and the correlation function is a full array correlation function; and the method further comprises: designating as a short array parameter data and perturbation data from the full array over a time period shorter than the time period of the full array and including the most recent data; obtaining a correlation function representing correlation between the short array parameter data and the short array perturbation data as for the full array correlation function; calculating one or more statistical coefficients of the short array correlation function and the full array correlation function that indicate the goodness of fit of the short array correlation function mathematical model and the full array correlation function mathematical model; comparing the statistical coefficients from the short array with the statistical coefficients from the full array to determine if the mathematical model fitting is improved for the short array compared to the full array; updating the full array by removing parameter data and perturbation data older than the oldest time in the short array if the comparison shows an improved fit, and by retaining all the full array data otherwise; and using the correlation function of the updated full array to calculate the correction factor. This enables a shortening or trimming of the data array to remove any old data that is less well correlated than newer data, thereby giving a more accurate value for the correction factor. Also, the amount of stored data is decreased, reducing the processing burden.

Following a trimming of the array, the statistical coefficients used to scale the magnitude of the correction factor can be statistical coefficients of the updated full array.

The time period for the short array can be determined to be a fraction of the time period of the full array determined in proportion to the goodness of fit of the full array correlation function mathematical model, such that a better fit yields a larger fraction and hence a longer time period. Other approaches to setting the short array duration can be used instead, including simply disregarding a fixed amount of data from the old end of the array.

The method may further comprise calculating one or more statistical coefficients of the correlation function that indicate the goodness of fit of the correlation function mathematical model; determining rates of change of the one or more statistical coefficients over the time of the data array; analysing the rates of change against one or more stability criteria to assess stability of the data over time; and if the stability criteria are met, calculating and using the correction factor, and otherwise obtaining more data until the stability criteria are met. These steps effectively allow the method to monitor its own performance, so that the correction factor is only calculated and applied under conditions where it is likely to provide a useful result, namely compensated parameter data that is genuinely improved over the original measurement.

The measurements may be obtained from media in a bioreactor during a bioreaction. The method is not so limited, however, and may usefully be implemented in the operation of other systems and apparatus.

The parameter may be refractive index and the perturbing influence may be temperature. The method is widely applicable to a range of other parameters and perturbations, however.

A second aspect of the invention is directed to a system comprising: a first sensor configured to collect data with respect to time of a signal representing a parameter modified by a perturbing influence; a second sensor configured to collect data with respect to time of a signal representing the perturbing influence; memory for storing the collected data in a data array; and a processor having program instructions configured to cause the processor to implement a method according to the first aspect.

The first sensor may be a refractive index sensor and the second sensor may be a temperature sensor.

The system may further comprise a bioreactor vessel, in which the first sensor and the second sensor are arranged to collect data from media in the vessel.

The processor may be further configured to use the compensated parameter data to generate one or more control signals to control operation of one or more components of the system. For example, the one or more control signals may comprise signals to control delivery of additive into the bioreactor vessel.

A third aspect of the invention is directed to a computer program product comprising a computer readable storage medium bearing instructions executable by a processor to implement a method according to the first aspect.

### BRIEF DESCRIPTION OF THE DRAWINGS

For a better understanding of the invention and to show how the same may be carried into effect reference is now made by way of example to the accompanying drawings in which:

FIG. 1 shows a schematic representation of an example bioreactor system in which embodiments of the present invention can be utilised;

FIG. 2 shows a schematic representation of a second example bioreactor system in which embodiments of the present invention can be utilised;

FIG. 3 shows a schematic representation of third example bioreactor system in which embodiments of the present invention can be utilised;

FIG. 4 shows a plot of signal and reference data acquired in a system such as those of FIG. 1, 2 or 3;

FIG. 5 shows a flow chart of steps in a method for compensating measured parameter data for noise according to an embodiment of the invention;

FIG. 6 shows a flow chart of steps in a routine for calculating data rates of change which may be included in the method of FIG. 5;

FIG. 7 shows a flow chart of steps in a routine for assessing the trend of data which may be included in the method of FIG. 5;

FIG. 8 shows a graph of statistical coefficients of modelled parameter and noise data that can be calculated during the method of FIG. 5;

FIG. 9 shows a flow chart of steps in an example routine for testing the stability of measured parameter and noise data which may be used in the method of FIG. 5;

FIG. 10 shows a flow chart of steps in a second example routine for testing the stability of measured parameter and noise data which may be used in the method of FIG. 5;

FIG. 11 shows a flow chart of steps in a routine for averaging stored parameter and noise data which may be included in the method of FIG. 5;

FIG. 12 shows a flow chart of steps in an example routine for calculating a correction factor for use in compensating measured parameter data for noise, for use in the method of FIG. 5.

FIG. 13 shows a plot of the measured data shown in FIG. 4 after compensation of the signal data using the reference data under a method according to embodiments of the invention; and

FIG. 14 shows a flow chart of steps in a routine for trimming the length of an array of stored parameter and noise data according to an embodiment of the invention.

### DETAILED DESCRIPTION

The invention will be described in detail with particular regard to a specific example system of a bioreactor controlled by measurement of refractive index to determined the timing of feed events (addition of nutrients), where the measured refractive index data is subject to interference or perturbation due to temperature changes. However, the invention is not limited to use with such a system, and other choices may be made as to the physical sensor type used for measurements, the system configuration and the detailed choices made in the software and algorithms. Also, the invention is applicable to the measurement, monitoring and adjustment of other parameters measured in other systems and subject to other perturbing influences.

In the following specific examples (to which the invention is not limited, however), the use of optical grating technology allows the measurement of both a refractive index signal (being data that reflects refractive index which is derived directly from optical changes within the fluid) and also thermal noise reference data (also measured optically, but lacking any dependence on refractive index) which can be used to compensate for temperature variation in the refractive index.

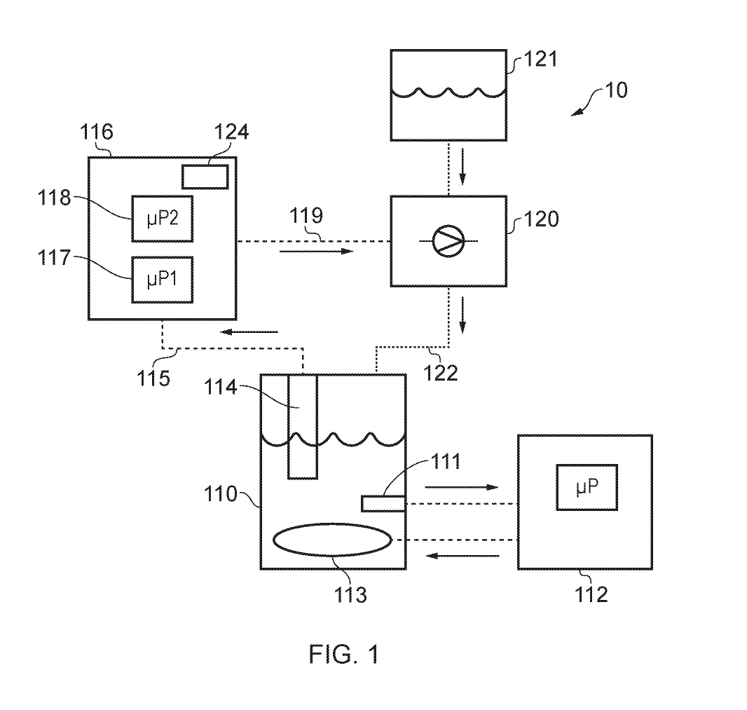

FIG. 1 shows a simplified schematic representation of an example bioreactor system 10 which can be controlled according to embodiments of the present invention. The various components are connected by tubing for the flow of additives such as nutrients (shown by the dotted lines) and by signal lines for wired or wireless transmission of signals representing measured parameters and control signals to control the process (shown by the dashed lines). The system includes a bioreactor vessel 110 which may be either single use or reusable, and may be made from any of various materials, including glass, metal and plastics. The volume of the reactor may range from microlitre volume in a micro-reactor up to tens of thousands of litres in a large volume industrial production reactor. The invention is applicable regardless of reactor size, so the reactor volume may be any volume in which the required bioprocess can be successfully operated. The temperature of the bioreactor contents (media) is to be controlled throughout the process to ensure proper growth conditions, and this is achieved by a temperature feedback loop. A temperature sensor 111 is positioned either within the reactor vessel 110 or in contact with the environment of the vessel to measure the temperature of the bioreactor contents and provide a thermal feed-back signal to a temperature controller 112 (which may be part of a larger control system). The temperature controller 112 then calculates the output power required from a heating element 113 inside the reactor 110 to supply heat to the reactor contents to maintain a target temperature for the process, and sends a control signal to the heating element 113 accordingly. A variety of control algorithms can be used to determine the power required to maintain a temperature set point; typically these will be a variant of P (proportional), PI (proportional-integral) or PID (proportional-integral-derivative) control loops. The temperature controller attempts to maintain a set temperature (which can change throughout the process), but due to the state of flux to which the reactor is exposed (including external laboratory temperature control, and gaseous and liquid additions to the contents) there will be fluctuations in the temperature of the media as the loop adjusts.

A second sensor 114 is positioned inside the reactor vessel for in situ monitoring of the contents of the bioreactor 110, by measuring or monitoring a selected bulk physical property from which it is possible to derive information about the media composition to as to determine the timings of additive events (feeding events). The bulk physical property may be refractive index as described in GB 1416233.3, in which case a suitable sensor device is the Ranger probe produced by Stratophase (www.stratophase.com). This device has two sensors embedded, Sensor 1 and Sensor 2, and is based on a planar Bragg grating evanescent sensor. Sensor 1 has a window over its grating, and the presence of the reactor media in the window when the probe is submerged modifies the response of the grating because the evanescent field of light propagating through the grating extends through the window into the media. The behaviour of the refractive index of the media can be deduced from the measured response. As is typical for refractive index sensors, the response signal emitted from Sensor 1 is a function of the temperature and stress of the sensor, as well as the refractive index of the media. In contrast, Sensor 2 comprises a grating without an overlying window, so that its response is a function of the temperature and stress but not of the refractive index of the media. According to the invention, the output of Sensor 2, which can be considered as a reference or noise measurement, can be used to compensate the output of Sensor 1, the signal or parameter measurement, by adjusting or correcting for temperature.

Although the Ranger probe provides a convenient tool for obtaining both the signal and reference measurements, it is not necessary for the two sensors to be integrated into a monolithic form. Beneficially, however, they are preferably of relative comparable thermal mass and in similarly representative locations within the reactor. The more similar and proximate the sensors, the better the resulting signal compensation will be. Using a refractive index sensor and a separate temperature sensor of, for example, the PT100 type arranged next to the refractive index sensor functions as well, but can increase the complexity of the system. The more divergent in thermal mass and positioning the two sensors are, the lower the accuracy of the resultant correction process.

The signal and reference measurements are obtained in situ from the probe 114, and pass by an electrical or optical cable or wireless link 115 to a controller 116. The controller 116 includes two microprocessors 117 and 118 programmed with software (or any equivalent data processing element or device implemented in hardware, software or firmware) to process the received information. The controller also has memory 124 accessible by the processors for storing the software, and for storing the measurement data received from the sensor 114 for retrieval by the processors 117, 118. The data is stored in the form of a historical array, with measured values of the signal and reference measurements stored with respect to time. Newly received values are added to the array, and old values may be removed from the array in accordance with operation of the compensation calculation as described later. A compensation microprocessor 117 performs the thermal compensation calculation described in detail below using the two sets of sensor data, namely the signal and the reference measurements to produce compensated or corrected refractive index data. A control microprocessor 118 implements a control algorithm utilising the compensated refractive index signal data for produce feed event control signals. A method for controlling bioreactor feed events using refractive index measurements is described in detail in GB1416233.3. Alternatively, processor 118 might be replaced with a data store or equivalent if the data is to be logged rather than immediately used to generate control signals. In the example of FIG. 1, the microprocessors 117 and 118 are illustrated as separate entities, but alternatively their functions could be incorporated into a single microprocessor, field programmable gate array (FPGA) or similar. The controller 116 will determine when a feed addition is to be made to the reactor 110 and sends a control signal, which may be electrical using for example analogue voltage or analogue, using a protocol such as Ethernet, I2C, USB, RS485, OPC, etc., by cable 119 or wirelessly to a pump 120 to control the pump's operation. The pump 120 is a pump system or valve which obtains from an additive reservoir 121 a required volume or quantity of additive (feedstock, etc.) and injects this amount of fluid or solid feedstock into the bioreactor via tubing 122. The additive will likely be at a different temperature to the media contents of the reactor 110 (due to the temperature controller maintaining a set-point for the media), so the introduction of the additive may perturb the thermal equilibrium of the media, creating a temperature variation. The refractive index of the media in the reactor will hence change not only because the composition has changed, but the temperature of the media as well. Such thermal fluctuations (which can also be caused by other external factors, for example laboratory air-conditioning) are not only a source of noise in the refractive index signal requiring compensation, but according to the invention, are also critically the source of the reference data used by an algorithm to generate the compensation data to correct the refractive index measurement.

The temperature controller 112, the pump system 120 and the process controller 116 may be integrated into the same physical enclosure or housing (for example, to form a control tower). Further, or alternatively, the microprocessors 117 and 118 may be disposed within a housing of the sensor 114 such that the sensor 114 is configured to generate and deliver control signals directly to the pump 120 without the need for a separate controller. The pump 120 and the reservoir 121 may together be considered as an additive supply mechanism. Such a mechanism might be configured in an alternative manner; the requirement for the mechanism is simply that it can automatically deliver a required quantity of additive into the bioreactor vessel in response to a control signal, be it liquid, solid or gaseous.

In many instances a bioreactor system will be considerably more complicated than the example illustrated in FIG. 1. For example, there may be a plurality of reservoirs and pumps to deliver several different feedstocks to the reactor vessel according to particular control signals generated by the control microprocessor 118 in response to the bulk property measurement. The present invention is applicable to any bioeractor system in which a parameter subject to noise is measured, for example for the purpose of generating a control signal, and a separate measurement of the noise is also obtained. The invention may also be applied in systems other than bioreactors, in which signal and noise measurements of this type can be recorded.

As explained further below, the compensation method of the present invention utilises correlation between the measured signal and the measured reference or noise. In some reactor configurations, the dominant thermal change in the reactor that produces the noise may have a very low time constant. Hence, while there are overall patterns in the process cycle the timing is generally not at a stable enough frequency for frequency domain techniques such Fourier analysis to be of use in removing the noise. There will typically be an overall fluctuation around the temperature set point of the reactor, however. In such systems, it can be beneficial to intentionally dither the temperature set point of the reactor around the intended set point. This effectively increases the noise, which is counter to conventional methods of removing thermal fluctuations by stabilising the temperature of a system, but can improve the method of the invention by enhancing the correlation between the two measurements. The temperature variance is set to be negligible to the organism being produced, but introduces enough of a swing in the temperature of the reactor at a higher frequency to accumulate useful data to implement the compensation. A typical variance would be for example ±0.2° C. on a set point of 38° C. The dithering may be a fixed frequency of oscillation, random cycling or anywhere between the two conditions. A benefit of an approximately random timing is that it avoids any accidental control loop beating of other parameters controlled (for example air conditioning in the laboratory). The same resultant effect can be achieved through miss-tuning a standalone P, PI or PID thermal feedback loop to have a degree of instability, such as using an overly large P value to intentionally overshoot the set point. In a system configured for an intentional controlled oscillatory temperature set point it is advantageous if the set point variation is controlled by the microprocessor controlling the thermal compensation (correcting the refractive index signal) or is linked in such a way that the thermal compensation microprocessor has information on the thermal perturbations. Note that temperature dithering is not essential, however. Indeed, the invention is equally applicable to a system that has no temperature control (in terms of FIG. 1, a system without the heating element 113, the temperature sensor 111 and the temperature controller 112).

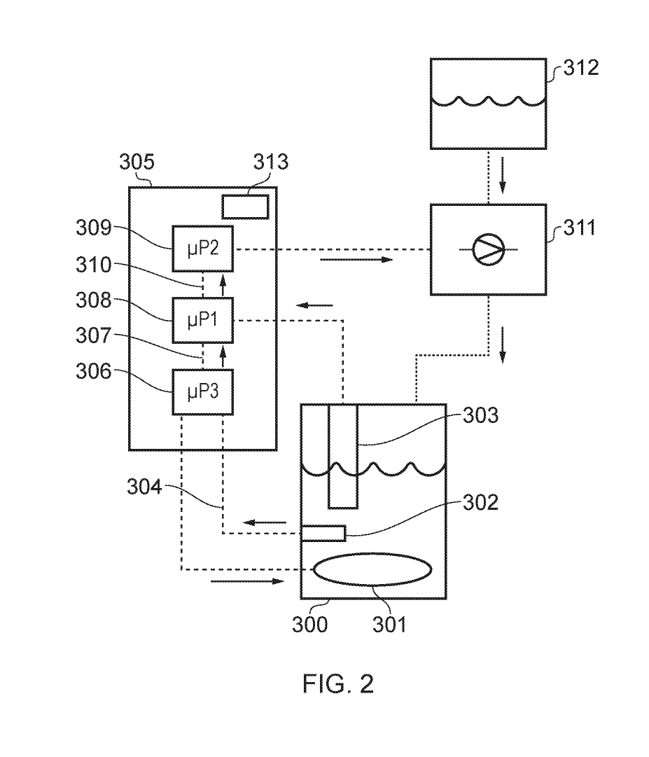

FIG. 2 shows a schematic representation of a system configured for linkage between the temperature control (possible including dithering) and the temperature compensation. The basic arrangement is similar to that shown in FIG. 1, with a reactor vessel 300, a heating element 301, a temperature sensor 302 and refractive index and reference/noise probe 303. However, in place of the temperature controller 112 of FIG. 1, the measured temperature signal 304 from the temperature sensor 302 is now connected to a unified control system 305. The unified control system 305 has a memory 313 for storing software for the processors and the historical data array, as in FIG. 1, and also has a reactor temperature control microprocessor 306 which controls the heating element 301 in response to the measured temperature signal 304. Temperature data 307 from the temperature control microprocessor 306 is sent to a thermal compensation processor 308 which also receives the signal and reference measurements from the sensor 303 (not necessarily in the same enclosure) via Ethernet, I2C, USB, RS485, OPC, etc., by cable, wirelessly, or as a separate function on the same processor. This enables the temperature data to be utilised in the compensation functionality. The functions of the thermal control microprocessor 306 and the thermal compensation microprocessor 308 can be incorporated with or without subsequent microprocessor function for example a feeding control microprocessor 309 generating feed event control signals for a pump 311 and a reservoir 312, either through an integrated control system sharing data or with all functions within a single microcontroller. In an integrated configuration the function of the two temperature sensors (i.e. the sensor 302 producing the temperature signal used to control the heating element, and the sensor 303 producing the reference measurement used to compensate the refractive index measurement) can be combined. For example probe 303 could be a refractive index sensor only with no sensor for the reference measurement, and the thermal compensation process can use the signal from the temperature control sensor 302 (such as a PT100 or LM35) so that the information in link 307 contains both the set point and the real time temperature.

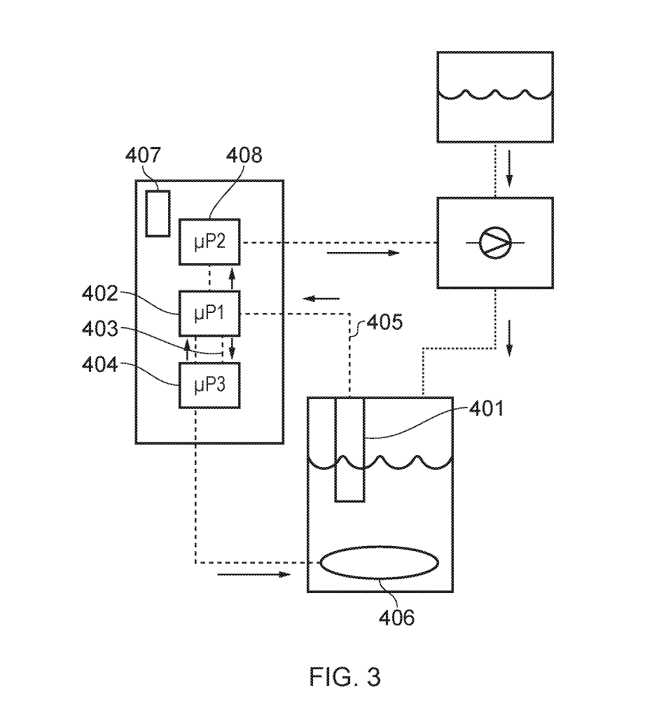

FIG. 3 shows a schematic representation of a further alternative integrated system. In this example, a combined probe 401 (for example the Ranger probe) is used, and the reference sensor thereon provides a temperature measurement 405 that is used as feedback 403 to a temperature control processor 404 to generate the control signal for the heating element 406, and by the thermal compensation processor 402 to calculate the compensated refractive index measurement. The refractive index-based signal data and the temperature-based reference data received from the probe 401 are stored to a data array in memory 407, as for the previous example systems.

In summary as regards temperature measurements, a system may comprise two separate temperature sensors to individually obtain a first thermal measurement for a feedback temperature control loop and a second thermal measurement being the reference or noise measurement used in the correlation process to calculate the refractive index compensation, or may comprise one single temperature sensor whereby the same thermal measurement is used for temperature control and compensation. Also, there may be just one temperature sensor whose output is used for compensation only, without any active temperature control.

Whichever type of bioreactor system and measurement arrangement is used, an embodiment of the invention requires the generation of two measurements to implement the correlation and subsequent correction/compensation. A first measurement (the signal) reflects the refractive index and includes temperature information as noise where temperature changes have perturbed the refractive index value, and a second measurement (the reference) reflects the temperature and contains the same temperature information. More broadly, the invention can also be applied to any system in which similar signal and noise measurements can be obtained.

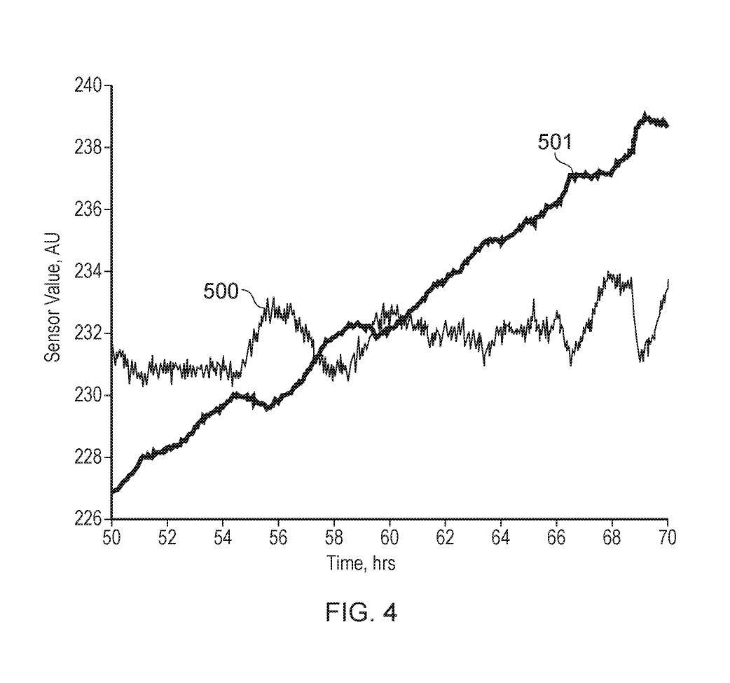

FIG. 4 shows example actual data collected by a Ranger probe with two channels (two sensors), over a time period of 70 hours of a bioreactor process run. Trace 500 is the measurement obtained from a Bragg grating sensor (Sensor 2) not exposed to the media in the reactor vessel and hence represents the reference temperature-based measurement. Trace 501 is the response measured from a Bragg grating sensor (Sensor 1) exposed to the media in the vessel, and hence represents the refractive index signal plus temperature perturbation. The reference trace 500 (called hereinafter “Sen2”) can be considered to be a function of the temperature of the probe, and in addition the strain observed in the sensor chip which is a function of factors including the age of the probe, number of sterilisation cycles in its history, the duration since immersion caused by water absorption on the glass surface or water absorption in the probe build materials. The signal trace 501 (hereinafter called “Sen1”) is a function of all the same stimuli as reference trace 500, and in addition of variations in the refractive index of the media caused both by composition changes and temperature changes, and any changes on the surface of the chip caused by fouling or erosion. There is a clear trajectory difference between the two traces, predominantly caused by composition change over the duration of the process run, but there is additionally a clear correlation between the two sensor traces at a higher frequency. For the purposes of illustration, the data in FIG. 4 has been selected as a good example of especially strong correlation between the measurements, but it is nevertheless apparent that the correlation is not possible to apply in a direct relationship, even if one was to compensate for the long term variation of trace 501 compared to trace 500. The compensation method of the present invention enables the correlation to be exploited via an indirect technique, however, so that the refractive index data represented by trace 500 can be corrected for thermal noise.

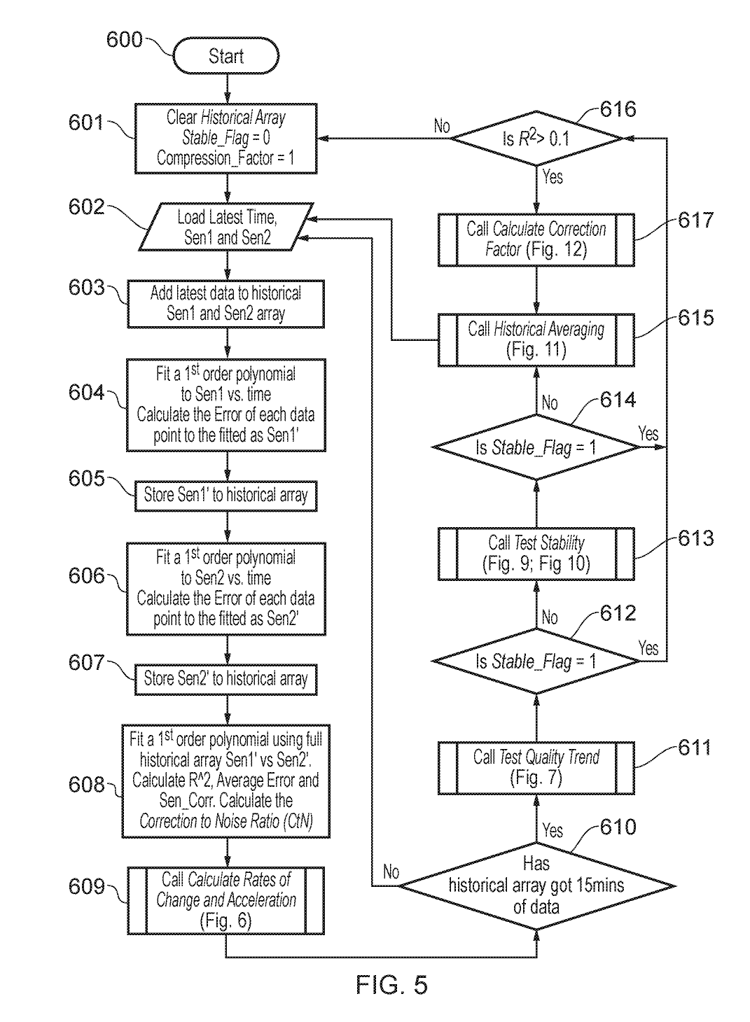

FIG. 5 shows a flow chart indicating steps in an embodiment of a method to correct or compensate measured data for a perturbation which uses that data plus a measurement reflecting the perturbing factor, for example the signal measurement Sen1 and reference measurement Sen2 of FIG. 4, which may be, for example, a measurement reflecting refractive index perturbed by thermal noise and a measurement including temperature information causing the thermal noise. The method is free-running and can be configured to require no user or control system interaction or knowledge of the process to operate. It may equally be applied to parameters and perturbing influences other than refractive index and temperature, and to systems other than a bioreactor with feed event control.

Some important points to appreciate about embodiments of the method are:
The compensation is based on correlating the two measurements, signal and reference.
Using the correlation, a correction factor is calculated and applied to the signal data to achieve the compensated result.
Statistics of the correlation are used to limit the amount of data used to calculate the correction factor when more recent data is found to be better correlated.
The method can include one or more tests based on statistics of the correlation, the outcome of which determine whether the compensation may validly be applied. Hence, if the correlation between the signal and the reference is poor, the method recognises that it is not useful to apply the compensation and the calculation will not be made.
It is possible to scale the correction factor using the statistics such that the magnitude of the correction factor depends on the size of error in the correction factor. In this way, the largest correction can be made in circumstances where the correction is deemed to be most valid and hence has a small error.

The method, implemented for example by software on a processor such as the compensation microprocessor 118 in FIG. 1, operates as a loop, shown in the flow chart of FIG. 5. Each loop corresponds to the collection of one pair of measurements Sen1 and Sen2, at a noted time. The start 600 of the loop can occur either at system power-up, when a sensor probe is attached to obtain the required measurement data, when a reactor ‘run’ (process to grow product) is about to be performed, or at any other time deemed useful by the user. It may be configured to begin automatically in response to a particular condition, such as attachment of the probe. At this initial point 601 a dynamic historical data array (such as comprised within the memory 124 of FIG. 1) is blanked (any existing data, such as was stored in a previous run, is deleted) and a variable Stable_Flag is set to 0 to indicate that there are known external reasons why a correction should not be applied at that time (in this case there has been no accumulation of data over time). The Stable_Flag is arranged to be set to 1 later in the loop if various conditions are met, thereby indicating that the compensation can usefully and validly be calculated and applied. Also, a Compression_Factor flag is set to 1, for possible use later in compression of the data array once it becomes large. Data collection then begins at 602. Measurements are made by the sensors in the probe, to obtain values over time (one measurement action per loop of the method) for, in this example, the refractive index signal plus temperature noise Sen1 and the temperature reference Sen2. As soon as new data is available synchronised (or near synchronised) values are taken for Sen1 and Sen2 at a known time. Note that this step 602 will also typically be the return point in the main program loop. The loop moves to step 603, where the newest values of Sen1 and Sen2 acquired in step 602 are stored in the dynamic historical array against the time of measurement, whilst any previously stored values are still preserved in the array. As the method operates, the loop will be implemented many times with new values of Sen1 and Sen2 stored each time. The values accumulate over time, and as explained later, the loop includes statistical tests to remove older values if they are found to be adverse to the quality of the final correction, and can include tests to determine when enough data has been collected for a useful compensation to be made to the signal measurement.

The methodology recognises that although the long term trajectories of Sen1 and Sen2 are independent, there are short term correlations, as apparent from the traces of FIG. 4. To normalise the impact of the long term trajectory differences, at the next step 604 a first order (or 1 degree) polynomial is fitted to the accumulated Sen1 data over time. This function approximates a base line for the longer term variation, against which the short term correlating fluctuations can be assessed. Any reasonable mathematical fitting technique can be applied to obtain the fitted function, such as a least squares fit or any of the more robust methods including least absolute deviation, least trimmed squares, least median deviation and the like. In a basic embodiment, a linear fit might be used. Furthermore, the fit does not have to be a first order polynomial fit, but could instead be a higher order function or equivalent or even a heavily averaged set of the same data. The purpose is to obtain a function that represents or models the long term drift of Sen1. Next, Sen1 is compared with the fitted function, and a signed error value (the difference between the data and the model at each point) is determined for each time in the data array between the measured Sen1 value and the fitted or averaged value. This error data is designated Sen1′. Moving to step 605 in the loop, the values of Sen1′ are added to the data array, overwriting any previous values entered (the fitted model and hence the error will evolve over time as Sen1 measurements accumulate, so the whole set of Sen1′ values, one value for each measurement time, is rewritten every loop.

At steps 606 and 607, these procedures are repeated for the Sen2 data. A function such as a first order polynomial is fitted to the Sen2 data. Note that for simplicity the same fitting technique can be used to fit Sen2 as was used for Sen1, but this is not essential. Different fits may be used if preferred. Then, signed error values (difference values) between the actual Sen2 data and the fitted curve are determined for each time point, and the error values are designated Sen2′ as the final action at step 606. At step 607, the Sen2′ data is stored to the data array with the Sen1′ data, overwriting any previous Sen2′ data.

The purpose of the steps 604 and 606 is to eliminate the effect of the different long term trajectories of the data, thereby normalising the data sets one to the other and allowing the two measurements to be directly compared so that correlation between them can be accurately identified.

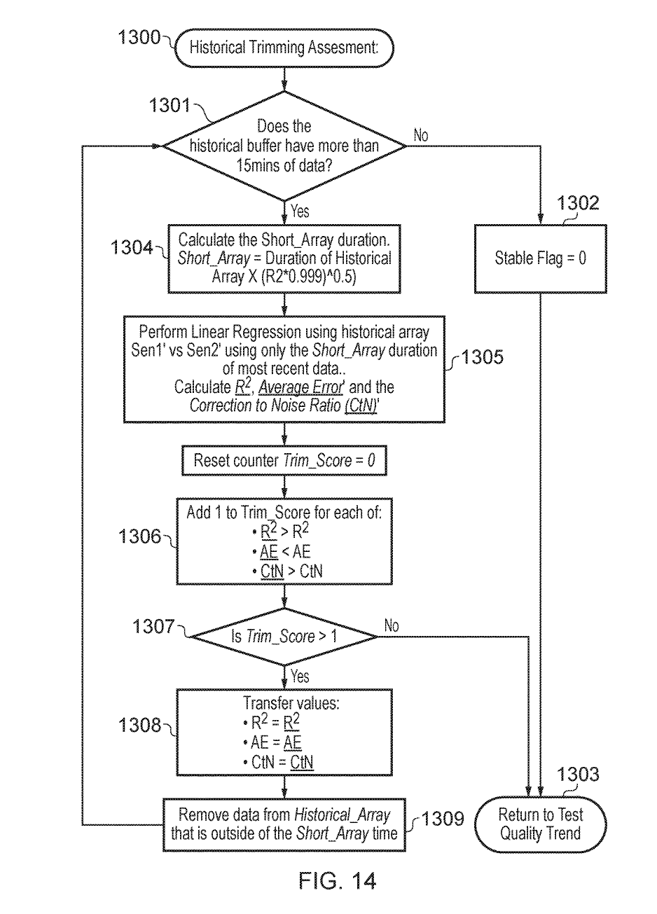

The method proceeds to step 608. Firstly, a correlation function is calculated by taking data from the full time period stored to date in the historical data array and performing a linear regression between the two sets of variables Sen1′ and Sen2′ by fitting a function to the Sen1′ data against the Sen2′ data. This fitted function is essentially a statistical model of the data, and is designated as the correlation function. This stage in the method defines the relationship between the two data streams (signal and reference/noise; in our example, refractive index and temperature) in both scale and confidence. The correlation function may be a first order polynomial, or could alternatively be a higher order polynomial or similar function. However, in some implementations a first order polynomial is attractive in that it has a computationally low overhead, and additionally can be directly meaningful in the subsequent analysis of the data, if the historical array duration is a correct length. This is explained in more detail later with respect to FIG. 14.

Then, a statistical analysis is carried out on the correlation function to obtain a set of statistical coefficients. The R² value (coefficient of determination or correlation coefficient) is calculated; as usual in statistics this is a number that indicates how well the data fit the correlation function. The higher the value of R², the better the data fit the correlation function, which is a statistical model of the data. Thus R² represents how good or bad the model is. Also, the Average Error is calculated; this is the sum of the absolute error of each point (being the difference between each data point and the correlation function) divided by the number of points. Additionally, the multiplier for the first degree of the polynomial fit (or whichever fit has been used to obtain the correlation function) is designated as Sen_Corr. Note that commonly used statistical alternatives could replace these coefficients if desired. For example, the adjusted R² value could be used in place of the R², or standard deviation could be used in place of the Average Error. These alternatives cause no detriment to the algorithm overall. The coefficients represent the validity of the correlation function, i.e. they indicate how well the correlation function models the data, and hence how well the Sen1 and Sen2 data are correlated.

An additional parameter to define the correlation validity is calculated at this stage, the Correction_to_Noise ratio, designated CtN. The CtN is calculated as the absolute delta of the fitted Sen1′ value over the Sen2′ span, divided by 2 times the Average Error. This yields a value greater than 0. The CtN is an expression for the noise on the regression (correlation function) versus the maximum change possible from the fitted relationship, a value akin to a signal-to-noise ratio. A value less than 1 indicates that the error in the correlation (i.e. the noise) is greater than the correction factor (i.e. the signal) and so is a poor regime to operate within. Data producing a CtN less than 1 will therefore not be able to be compensated very well by the remainder of the method. A value of 2 indicates that the correction factor is twice the error and so is a good regime to operate within; the data will be able to be usefully compensated by the method. The values of R², Average Error, Sen_Corr and CtN are stored in the historical array for the latest timestamp. Since they are calculated from data over the full array available at the time, they represent the quality of the correlation up to that point. As the method loop repeats, new values are calculated and stored for each timestamp.

The method moves to step 609. The change over time of the statistical coefficients is of interest in determining if the correction can be validly applied, so taking the R², Average Error, Sen_Corr and CtN values stored in the array, a routine is called: “Calculate Rates Of Change and Acceleration”.

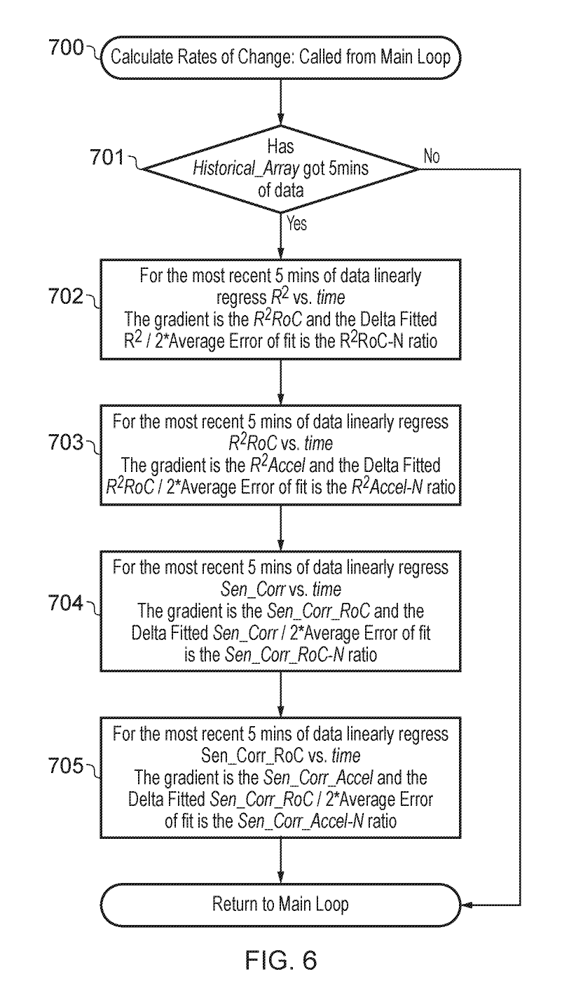

FIG. 6 shows a flow chart of the Calculate Rates Of Change and Acceleration routine. The purpose of the routine is to investigate how the values of R² and Sen_Corr have changed over a recent time period, by calculating rates of change (derivatives). In the embodiment of the method illustrated in FIG. 5 this routine is called every time the method loops, that is, for each newly stored pair of Sen1 and Sen2 values. Alternatively it may be called a slower rate to the raw data update, to reduce the amount of processing. The routine initialises at step 700, and firstly performs a test at 701 to determine if there is 5 minutes of data in the historical array. If there is not this much data, the routine returns to the main loop of FIG. 5, and will then be directed back to step 602 to acquire more Sen1 and Sen2 data. If there is 5 minutes or more of data available in the array, the routine progresses to step 702. The time constant of 5 minutes is merely an example, and has been chosen in this embodiment because the inventors have observed through practice that this length of time allows low noise rate-of-change calculations to be performed without significantly impacting the response time of the system. Other values are equally applicable and may be set to reflect the nature of the system that is used to collect the data and the rate of data collection. A finite time duration is required, however, so that behaviour over time can be assessed. In step 702 the processor accesses the most recent 5 minutes of data from the historical array. Taking the R² data, the most recent 5 minutes of values are linearly regressed using a first order polynomial fitted using a least squares fit with respect to time. The gradient, or first degree coefficient, of the fitted line is the rate of change of the R² value, and is labelled as R²_RoC, and in stored in the historical array against the current time stamp. Then, a “signal to noise ratio” for this R² rate of change is calculated in the same way as the earlier CtN, being the delta of the fitted R² values over the span of the linear regression divided by 2 times the average error of the fitted line, to yield the R²_RoC-N_Ratio. This is stored in the array also.

Moving to step 703, identical or similar calculations are then performed using the last 5 minutes of the R²_RoC values in the historical array (including the latest one from step 702) to yield the R²_Accel (the first degree coefficient or gradient of the fitted line) and the R²_Accel-N_Ratio. In steps 704 and 705, these calculations are repeated for the most recent 5 minutes of Sen_Corr values, yielding Sen_Corr_RoC, and Sen_Corr_RoC-N_Ratio, and then for the most recent five minutes of the Sen_Corr rate of change, to yield Sen_Corr_Accel and Sen_Corr_Accel-N_Ratio. As described, the method utilises linear regression as the regression technique but there are several alternative methods that could be employed to determine for rates of change values. For example, the difference between two consecutive data points, the difference between two sets of averaged data points, or two rolling averages with different buffer sizes all yield rates of change information and could be used for the calculation.

In summary the Calculate Rates Of Change and Acceleration routine returns R²_RoC, R²_RoC-N_Ratio, R²_Accel, R²_Accel-N_Ratio, Sen_Corr_RoC, Sen_Corr_RoC-N_Ratio, Sen_Corr_Accel, and Sen_Corr_Accel-N_Ratio values calculated over the previous 5 minutes of data. The values are all stored in the historical array against the current timestamp. Upon completion the routine returns the method to the main program loop of FIG. 5.

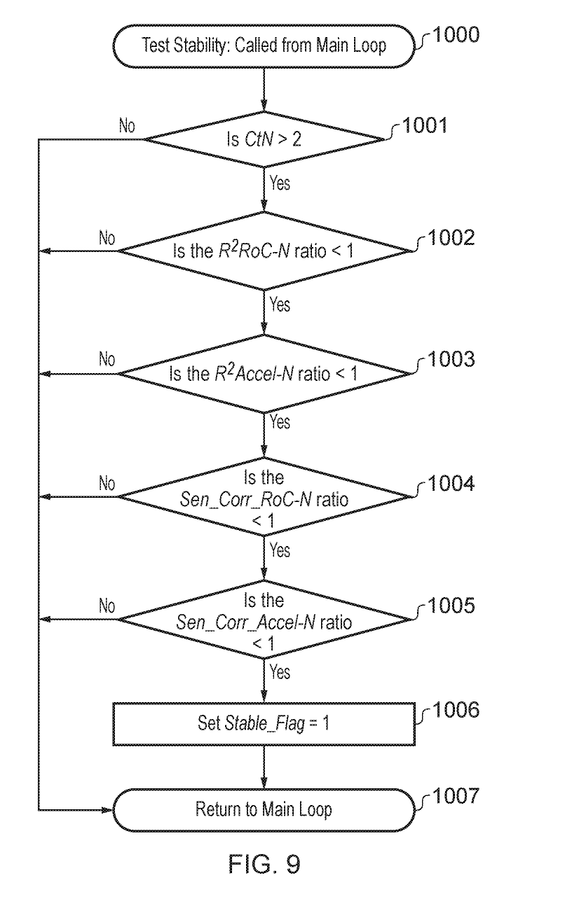

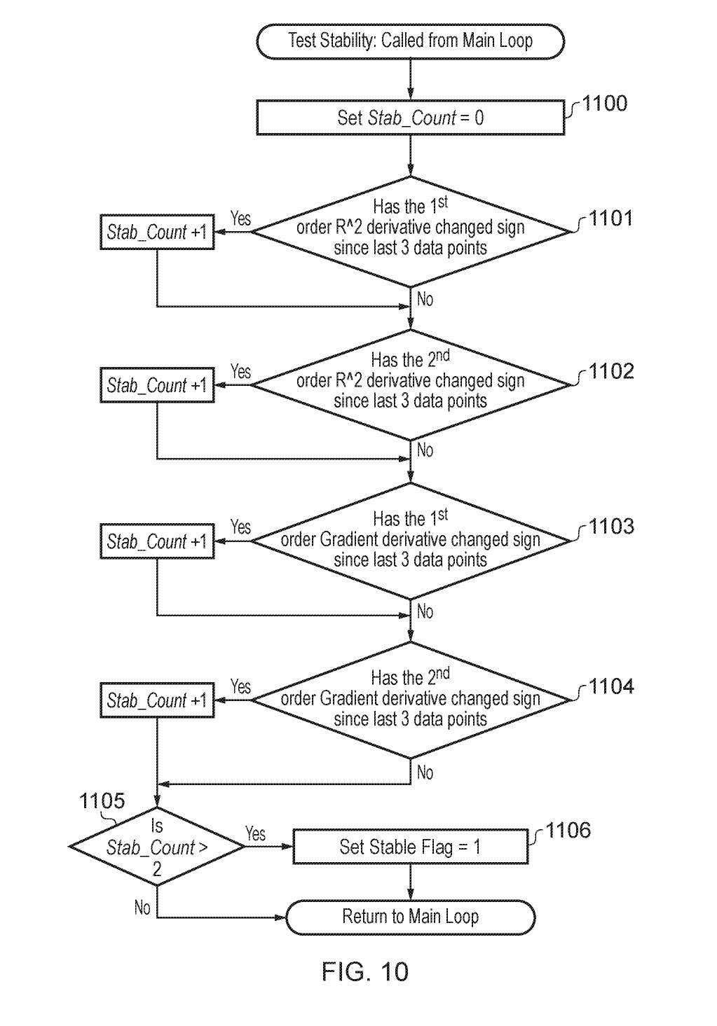

The main loop then advances to a logic gate 610, which represents an assessment of the amount of data currently stored in the historical array (buffer size) against a predetermined minimum duration of buffer deemed appropriate for the process to determine the quality of the eventual correction. A useful feature of the compensation method according to the invention is that it is possible, using the various statistical outputs, to determine if the eventual correction will be good or poor; if it is poor there is likely little point in applying it. The application can instead be deferred until the quality improves. To determine this, however, a minimum amount of data is required since the determination relies on changes and trends in the data over time, and a short window often will not yield an accurate view. Consequently, in the logic gate 610, the size of the buffer is examined to determine if there is at least 15 minutes of data present in the array. In this embodiment the 15 minute duration is selected as being an integer multiple of the 5 minute time test (step 701 of FIG. 6) used when calculating the derivative information in the Calculate Rates Of Change and Acceleration routine. However, from a consideration of the data rates and likely response times of the system (thermal response in the present example) the skilled person could determine an alternative value. A purpose of the delay afforded by this step is to avoid a low number of data points, or a small window of time where statistically valid high accuracy values might be determined for all the parameters, but would not represent the general interaction of the two data sets Sen1 and Sen2 but only small transient parts of data. Practically, in the example system a 15 minute delay enables the statistical routines to have sufficient data to properly assess stability of the process (described later with respect to FIGS. 9 and 10). Considering logic gate 610, if the duration of the historical buffer is less than 15 minutes it is determined that no correction will yet be made to the Sen1 data and the main program loop returns to collect new data in step 602. Once the historical buffer has met the condition of at least 15 minutes of data the method advances to step 611 where a routine Test Quality Trend is called.

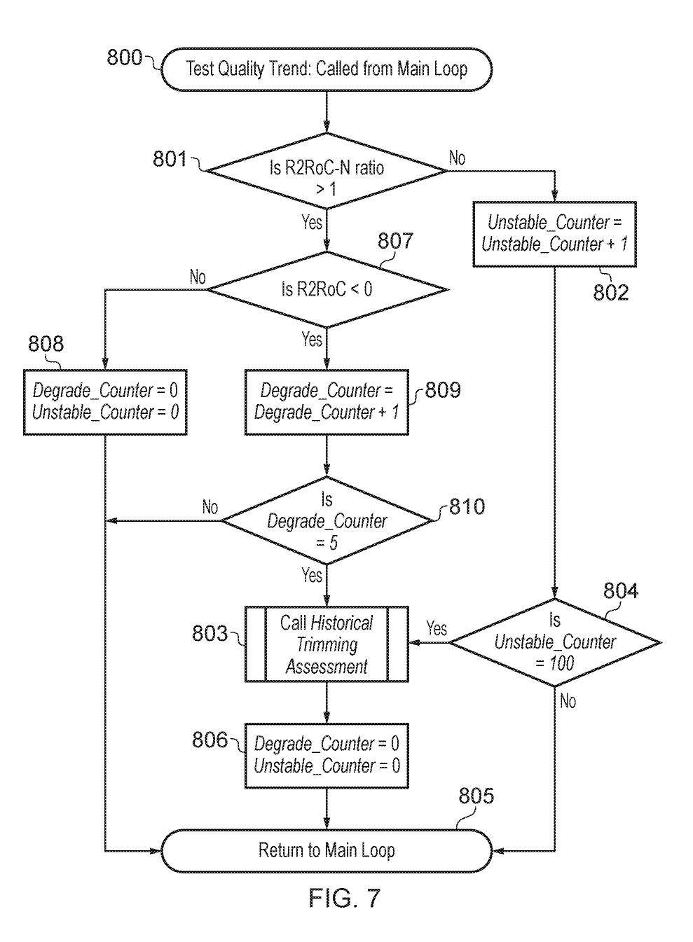

FIG. 7 shows a flowchart representing the steps of the Test Quality Trend routine. Recall that in step 608 of the main method (FIG. 5), various statistical coefficients were calculated, including the R² value, or correlation coefficient, which indicates how well the data fit the correlation function. A new value of R² is calculated for each loop of the method and stored in the data array against the current timestamp, so it is possible to track R² determining if over time, it is improving (value increasing) or getting worse (value decreasing). The Test Quality Trend routine is a function configured to achieve this tracking, and uses the rates of change calculated in the Calculate Rates of Change routine (FIG. 6). The nature of the trend is then used in the routine to determine if it is appropriate to trim the amount of data in the historical array; this can be done to remove older data representing unstable behaviour of the system, and also to keep the size of the array within manageable limits. The Test Quality Trend function is called from the main loop in step 800, and an immediate assessment is made in step 801 of the variance of the R² by looking at the R²RoC-N ratio which was calculated in step 702 of the Calculate Rates of Change routine. If the R²RoC-N ratio is less than 1, then the trend is determined to be stable, or within the noise. To reflect this, a counter named Unstable_Counter is incremented by 1, in step 802. The function of the Unstable_Counter is to ensure that a trimming of older data from the historical array (described further later) is performed even with little signal but at a reduced frequency (i.e. not every time the main method loops) so as not to perform excessive processing loops performed even with little signal. To implement this, the next step is 804 in which the value of the Unstable_Counter is tested. In this example, the test is whether the counter has yet reached 100, so that the trimming is performed only once for every 100 cycles of the method. Other values can be chosen instead, reflecting the nature of the system and the available processing power. If step 804 shows that the Unstable_Counter has not yet reached the set limit (100 in this example), the routine is exited in step 805, and returns to the main loop of FIG. 5. If step 804 shows that the Unstable_Counter has reached 100, the routine proceeds to step 803 where a Historical Trimming Assessment function is called to trim the size of the historical array. This will be described in more detail later, with respect to FIG. 14. After the trimming, the Unstable Counter is reset to 0 (step 806) to show that the trimming has recently been implemented to prevent unnecessary repeated calls to the trimming function. A further counter called Degrade_Counter used elsewhere in the routine is also set to 0 in step 806. The routine then returns to the main loop at step 805.

Returning to the test in step 801, if the R2RoC-N ratio value is greater than 1, the routine proceeds to step 807 to additionally test the value of the R2RoC that was calculated in step 702 of the Calculate Rates of Change routine (FIG. 6). If the R2RoC is positive (greater than zero), then the value of R² must be increasing with time. This indicates that the trend of the data is good, the modelling of the correlation is accurate, and the final corrected calculation for Sen1 will be worthwhile. In this case all the data in the historical array is valuable and there is no need to trim the historical array. Hence the so the counters Unstable Counter and Degrade Counter are reset to 0 in step 808 and the routine returns to the main loop in step 805. If instead the R2RoC is found to be negative (less than zero) in step 807, R² must be decreasing in a statistically relevant way, indicating a poor trend in the data. To reflect this, the Degrade_Counter is incremented by one in step 809, and then its value is tested in step 810. The Degrade_Counter acts as a delay before calling the Historical Trimming Assessment to reduce the frequency of calls to the trimming routine as it is relatively computationally intensive and successive incremental calls are unnecessary to a good final result. Thus, only when a set number of reductions in the R² value, reflected in successive negative values of the R2RoC, have occurred is the array trimming implemented. In this example, the number is chosen as 5 (other values may alternatively be chosen, having regard to the amount of data processing required and overall efficacy of the method) so that step 810 tests if the Degrad_Counter equals 5. If the counter does not yet equal 5, the routine returns to the main loop at step 805. If the Degrade_Counter has reached 5 then the Historical Trimming Assessment is called as before in step 803. Subsequently the Degrade_Counter and the Unstable_Counter are set to zero in step 806 as before, the routine then returns to the main loop in step 805.

Back in the main loop, following the Test Quality Trend function of FIG. 7 a logic test is applied in step 612 to identify if the Stable_Flag has a value of 0 or 1. This will have been set to 0 at the outset of the method overall, in step 601, and in a first pass of the main loop, nothing thus far will have changed that value. This reflects that only one pair of Sen1 and Sen2 values will have been measured and stored so that no correction is yet possible. A Stable_Flag value of 1 is required for the main loop to reach a calculation of the correction, and on a first pass this is not possible. Downstream from step 612, various mechanisms are implemented which can result in the Stable_Flag being set to 1 (reflecting that suitable data is available to implement a correction), so that in subsequent passes of the main loop the test in step 612 might be met. In general, if the Stable_Flag is set to 0 when the main loop reaches step 612 the algorithm implemented by the main loop has not thus far been deemed to be stabilised for a good result. The loop thus proceeds to step 613 where a Test Stability function is called to determine if adequate stability has been reached. If instead step 612 finds that the Stable_Flag is set to 1, the main loop proceeds via a different branch to a potential correction calculation using the current stable data, described further later.

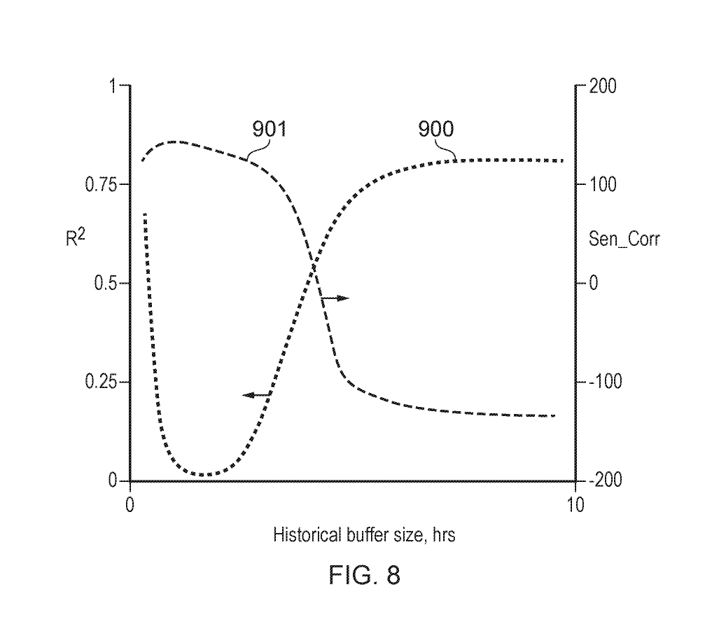

As the main loop repeats, adding a pair of Sen1 and Sen2 values each time, the historical buffer increases in length. The R² value and Sen_Corr value can change dramatically as the new data is incorporated into the historical array and, potentially, into the compensation calculation. FIG. 8 shows a plot of how the R² and Sen_Corr values can vary over time. In this example, the R² value 900 (dotted line) oscillates from an initial mid-range value to a low stable value to a later higher stable value. Likewise the Sen_Corr 901 (dashed line) initially starts with a fairly variable and inaccurate value when there is a small amount of historical data, before changing and finally stabilising as the buffer increases. Initial values can be wildly wrong, including having the wrong sign. The correction method of the present invention is improved by the use of stable and accurate data, so the Test Stability function is applied to determine if the data is stable enough to ensure that the Sen_Corr (used in the eventual correction calculation) is close to a proper value for a good end result. It is worth noting that in this methodology there is no single correct value for Sen_Corr, only the best that can be calculated at the time, where this value is expected to change significantly over the course of a process (such as a bioreactor run), as indicated by the example data in FIG. 8 (where the data are simplified ideal cases for the purposes of illustration). The time required to achieve a stable value varies significantly with process type and equipment location, so it is desirable to periodically test for stability so that the final correction calculation is only implemented when it will yield a good result. A variety of routines can be implemented to test for stability.

FIG. 9 shows a flowchart of a first example of a routine suitable for the Test Stability function. The process detailed in FIG. 9 is one of several possible processes that can detect stability in such cases; alternatives can readily be designed with variant tests or numbers of tests. The example in FIG. 9 is designed to make minimal assumptions about the time response and instability of the process; indeed there are no system settings required to be fed into the logic for implementing the routine. The routine is called in step 1000 from step 613 of the main loop, and starts with a first logic test at step 1001. This test asks if the Correction to Noise (CtN) ratio calculated in step 608 is greater than 2, which (as discussed above with regard to step 608) indicates that the correction signal is well in excess of the noise, so that data correction could be effective. This threshold value of 2 is arbitrary and other values might be chosen, but 3 dB (corresponding to a value of 2) is commonly taken in many fields as representing a good signal to noise ratio. If the CtN ratio is less than 2, the test at step 1001 fails and the routine is immediately exited at step 1007 to return to the main loop so that more Sen1 and Sen2 data can be collected. If the CtN ratio is greater than 2, the Test Stability function can proceed. In this example, there are four further tests, all of which must be passed to enable the data to be deemed stable. The “signal to noise” ratios of the rate of change and acceleration values calculated in the Calculate Rates of Change routine (FIG. 6) are analysed to define when the values are effectively zero (i.e. if the fitted signal change is less than 2*Average Error on the actual signal then it can be considered as 0), and this is determined with a ratio value less than one. So, in step 1002 the R2RoC-N ratio is tested to see if it is less than one, in step 1003 the R2Accel-N ratio is tested to see if it is less than one, in step 1004 the Sen_Corr_RoC-N ratio is tested to see if it is less than 1 and in step 1005 the Sen_Corr_Accel-N ratio is tested to see if it is less than one. If any of these rate of change or acceleration parameters are not zero (or less than 1, in terms of the test), the values for R2 and Sen_Corr are not considered to be stable and the routine exits at step 1007 to return to the main loop. If all the ratios are less than 1, the Stable_Flag is set in to one in step 1006 to indicate that a stable condition has been met and the loop exits at step 1007 back to the main loop. Hence, the overall effect of the Test Stability routine is to set the Stable_Flag from zero to one if and only if a set of noise ratios which reflect the stability of the data over time are effectively zero. A value of one for the Stable_Flag allows the main loop to proceed to a potential correction calculation.

FIG. 10 shows a flowchart of a second example of a routine suitable for the Test Stability function, as an alternative to the routine of FIG. 9. In the FIG. 9 routine, a number of values are tested so that if all tests are passed the Stable_Flag is set to one. In contrast, the FIG. 10 routine tests a number of values and sets the Stable_Flag to one if a proportion of the tests are passed. This is achieved using a Stability Counter (Stab_Count) which is incremented for each test passed, and then evaluated to determine if the Stable_Flag can be set to one. The function is called from the main loop, and in a first step 1100 the Stability Counter is set to zero. Then four tests are performed sequentially. At step 1101 the rate of change of the R2 value (i.e. R²RoC, being the first order derivative of R²) is tested to determine if its value has changed sign over the last three data points (corresponding to the last three measurement times). In step 1102 the same test is applied to the second order derivative of R², or R²Accel. In steps 1103 and 1104 the same test is applied respectively to the first and second derivatives of Sen_Corr, namely Sen_Corr_RoC and Sen_Corr_Accel. All these values were previously calculated in the Calculate Rates of Change routine shown in FIG. 6. If one of the derivatives is zero, it is very likely to be constantly changing sign due to the noise on the real data. This effect can be used to determine when a parameter is at zero without taking into account the noise of the measurement. For every one of these tests 1101, 1102, 1103, 1104 that results in a positive, i.e. there has been a sign change, the Stab_Count is increased by one. After all the tests, the Stab Count itself is tested at step 1105. If its value is greater than 2 (indicating that at least three of the four tests were passed), then the process can be considered to be stable and the Stable_Flag is set to 1 in step 1106. If the Stab_Count is not greater than 2, the Stable_Flag is left at zero. Finally, the routine returns to the main loop.

The Test Stability functions in FIGS. 9 and 10 can be implemented with a smaller or greater number of tests or with a lower or higher number of tests required to be passed. For example, the FIG. 10 routine might only test the R² derivatives or the Sen_Corr derivatives but not both. While decreasing the required computation, fewer tests increases the risk of falsely identifying when a process is stable. Also, any other test yielding the same information, namely whether there is stable data available for a correction calculation, can be used instead or as well.

Returning to the main loop in FIG. 5, following the Test Stability routine of step 613, there is a second logic gate 614 that examines the value of the Stable_Flag. During the Test Stability routine the Stable_Flag may have had its value set to one, in which case the main loop proceeds down a path leading to potential correction calculation, as will be described later. If the Stable_Flag is found to still be zero, the loop proceeds to step 615, which calls a Historical Averaging routine. The Historical Averaging is optional, as it does not improve the accuracy of the correction calculation. If it is not included, the main loop will return directly to the step 602 to acquire the latest Sen1 and Sen2 values; otherwise it returns to step 602 via the Historical Averaging.

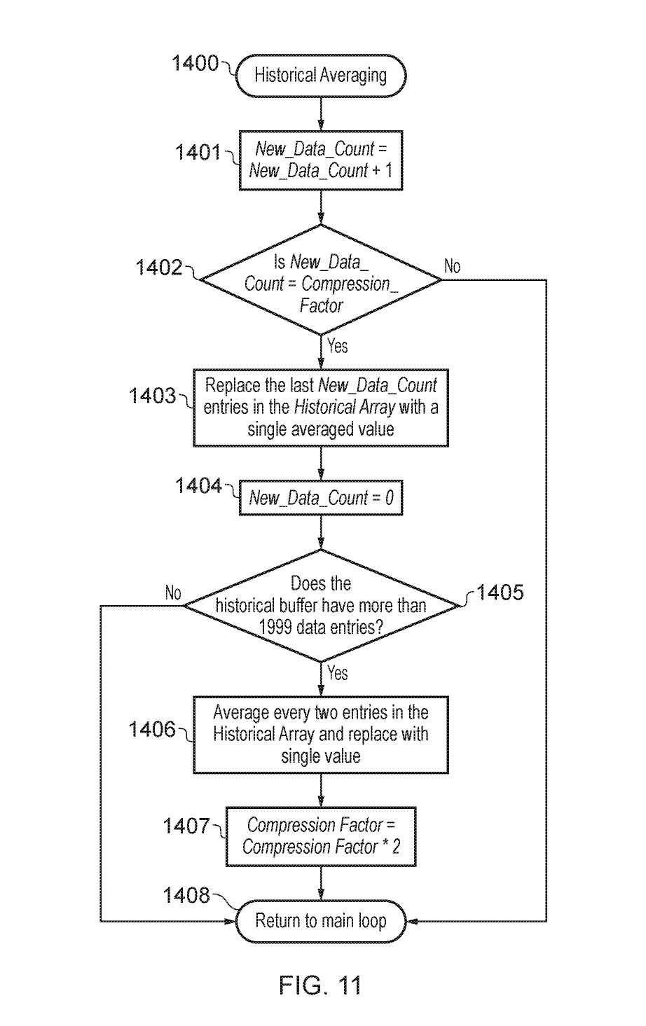

FIG. 11 shows a flowchart of the steps in the Historical Averaging function or routine. The Historical Averaging is designed to assist the ability of the historical array to gain an un-constrained amount of data as required for the correlation equation. It is entirely possible that some monitored processes (particularly slow ones) gain from having very long array durations, for example days or weeks of data, where it would be useful to reduce the total number of data entries over that time to reduce data processing requirements. The Historical Averaging function limits the number of array entries in the Historical Array enabling the maximum computational burden to be limited, within imposed limits on the total history that can be utilised. Effectively, the number of data entries covering a given time period is reduced while preserving the length of the time period. The routine detailed in FIG. 11 is one of several ways of reducing the data set without unduly degrading the data in the array. Other routines with a similar result might be used instead. Once initialised in step 1400, a value of 1 is added to a counter variable New_Data_Count in step 1401. This counter, which shows how many times the Historical Averaging routine has been run without any compression or averaging having been performed, allows the most recent data points to be compressed or compacted as quickly as possible to match the periodicity of the data in the rest of the Historical Array (which may have been previously reduced compared to the periodicity of data acquisition). In step 1402, the New_Data_Count value is compared to the value of the Compression_Factor, which was originally set to one on starting the main loop in step 601, but has possibly incremented in any previous operations of the Historical Averaging function. The Compression_Factor indicates how much the data has been compressed over the length of the array, in terms of how many original data points are now represented by one data point in the array after historical averaging has been applied. If the New_Data_Count and the Compression_Factor are not equal, the Historical Averaging routine is exited in step 1408, and the method returns to the main loop to acquire more data at step 602. If the New_Data_Count is equal to the Compression_Factor, then in step 1401 the most recent New_Data_Count value number of entries in the array are replaced with a single averaged value 1403. This brings new data into the same periodicity as older data which has already been compressed. Following this, at step 1404 the New_Data_Count counter is reset to 0. After these data changes have been updated to the array, in step 1405 an assessment is made of the size of the array. If the array contains, for example, at least 2000 data entries or sets, then the routine proceeds to step 1406 where every two adjacent entries in the array are replaced with a single average value 1406. The number of entries is thereby reduced by half, but the duration of the array remains the same. The data is hence compressed. To reflect this, the Compression_Factor value is doubled to reflect the new compression level, in step 1407. Then the routine is exited at step 1408, and returns to the main loop. If in step 1405 the array has less than 2000 data points then the routine is directly exited back to the main loop at step 1408. The routine may readily be modified to achieve a similar effect. For example, the minimum of 2000 data entries may be made a larger or smaller value according to preference. The compression rate applied in step 1406 may be different from two, for example three entries might be averaged to obtain a new single value. Other date averaging and compression techniques might also be used as desired, such as stripping out data without averaging.

Returning to FIG. 5 showing the main loop, if the Stable_Flag is found to be equal to one in either step 612 or 614 (so that the data and the process producing it have been deemed sufficiently stable to make correction worthwhile and accurate), the loop proceeds directly to calculation of the correction, via one final check in step 616. This check places a limit on the R² value, by testing if the R² is less than 0.1. If it is, then the loop is completely restarted by being returned to step 601 where the whole historical array is erased and the Stabilty_Flag and the Compression_Factor are reset respectively to 0 and 1. This check is designed to only become active if there is a big change in the media composition leading to erroneous readings from the sensor (making the data fitting poor and producing a low R² value), when it becomes possible that the validity of the historical array is beyond the ability of the Historical Trimming Assessment function to recover (described further later with regard to FIG. 14), so that the data should be abandoned. The threshold of 0.1 is arbitrarily chosen; the check would work with any value greater than 0 and less than 1, with the frequency of reset determined accordingly. In practice, a typical useful range is 0.1 to 0.4. Also, this function is not critical and the check can be omitted, as it will likely rarely be failed. Its main impact is to speed up recovery from large changes, which might be considered desirable in some systems. If the R² value is greater than the threshold (0.1 in this example), the check of step 616 is passed, and the main loop proceeds to step 617 where a Calculate Correction Factor function is called.

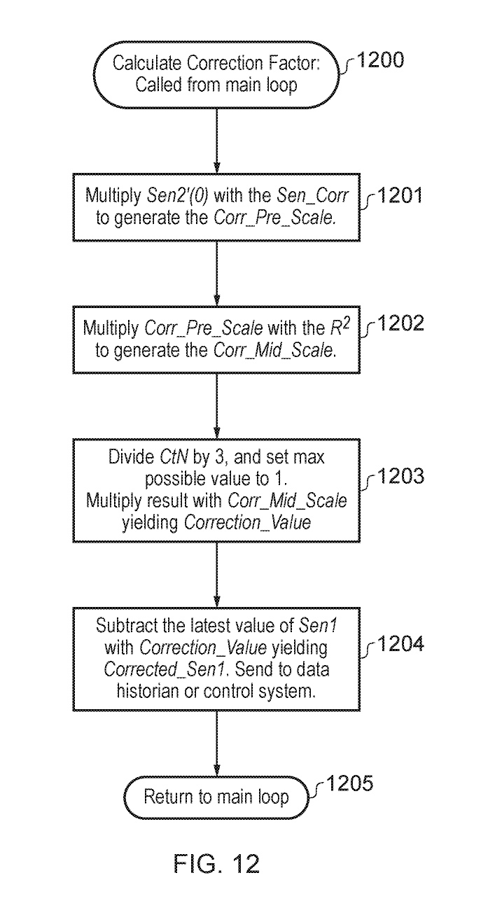

FIG. 12 shows a flowchart of steps in a routine for calculating a correction factor to be applied to Sen1, using the stored data and previously calculated coefficients once the data has passed stability tests. Once the Calculate Correction Factor function is called at step 1200 after the R² threshold test in step 616, it proceeds to step 1201 where the value of Sen2′ (calculated in step 606) for the most recent time stamp is retrieved from the data array, designated as Sen2′(0), and then multiplied with the current value of Sen_Corr. The result is labelled as the Corr_Pre_Scale, that is, the correction value before any scaling has been applied. The Corr_Pre_Scale is the direct perturbation expected on Sen1′ caused by the observed change in Sen2′. In a process with no errors, this value may simply be directly subtracted from Sen1 to give a corrected result compensated for the noise. In the main example, the refractive index measurement is thereby corrected for the thermal noise. This works also in a real system with errors, but can increase the average noise in the result since not all the perturbations in Sen2 are reflected in Sen1. To address this, a series of additional scaling steps are proposed. These are applied to reduce the magnitude of the Corr_Pre_Scale based on the confidence of the statistics in the correlation calculation. So, in step 1202, Corr_Pre_Scale is multiplied with the R² value. The R² value has a range of 0 to 1 range, so naturally lends itself to acting as a scalar. Alternatively, since there is no pure mathematical link to the scale function, instead of using the R² directly one can alternatively multiply by between 2 and 3 times the R² value (capping at a maximum value of 1). The result of this R² scaling is called the Corr_Mid_Scale (the correction value midway through a scaling procedure). The next step 1203 derives a second scaling factor, based on the CtN (noise of the compensation, from step 608). To achieve this, the CtN is divided by a scalar value, for example 3 although other values might be preferred, and the result capped at a maximum value of 1. The scalar value can be tuned for a particular measurement type with typical values ranging from 2 to 10, but 3 has been found to give good results in practice, having the effect that only a CtN of less than 3 will result in any damping. Also, different scalar functions can be used to modify the CtN, such as (CtN−1)/2 limited to between 0 and 1, and will work equally well. The objective is to bias the strength of the correction with the confidence in the correction, so any scaling that achieves this may be implemented. The modified CtN value is multiplied with Corr_Mid_Scale to produce the final scaled correction value, designated the Correction_Value. Finally, in step 1204 the correction value is applied: the Correction_Value is subtracted from the value of Sen1(0) to get the Corrected_Sen1.In practice, steps 1201 to 1204 can be performed simultaneously as a single calculation; in FIG. 12 the steps are broken out for clarity. The Corrected_Sen1, being the measured signal corrected for noise, for example refractive index corrected for temperature variation, is published to any process that requires it such as a control system or data archive. In the example bioreactor systems of FIGS. 1 to 3, Corrected_Sen1 is the corrected refractive index measurement from which control signals for feeding events are determined by the control microprocessor 118, 309, 408, the corrected measurement having been calculated by the compensation microprocessor 117, 308, 402.

Finally, the Calculate Correction Factor routine returns to the main loop in step 1205. The Historical Averaging routine of step 615 (FIG. 11) may optionally be executed before the main loop returns to step 602 to acquire the next pair of Sen1 and Sen2 measurements, following which the main loop repeats. Hence, for every pair of Sen1 and Sen2 measurement values acquired, the curve fitting and statistical analysis is performed over all data in the array to that time, the stability and quality tests are applied, and if these are passed, the correction factor is calculated and applied to correct that particular Sen1 value. The Sen1 signal is thereby incrementally corrected with each pass of the main loop. An initial delay (15 minutes in the examples) is included before the tests and correction calculation begin to be executed to allow adequate measurement data to accrue in the system memory for the calculations to be worthwhile.

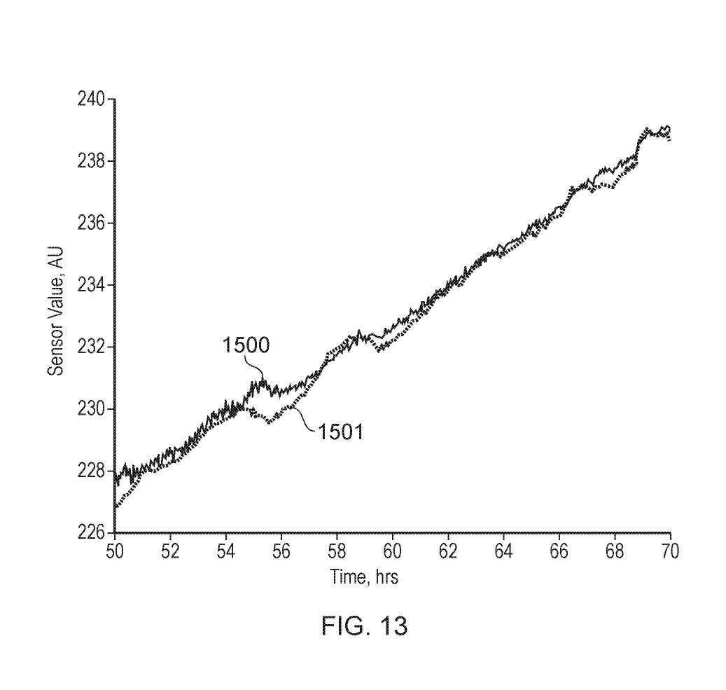

FIG. 13 shows a plot of corrected data compared to the original noisy signal, plotted over 70 hours. The corrected data 1500 (solid line) has a moderate increase in high frequency noise, but the majority of the slower fluctuations (over time scales of hours) visible in the original data 1501 (broken line) have been reduced or removed entirely.

Recall that the Test Quality Trend routine at step 611 of the main loop, shown in detail in FIG. 7, includes within it a Historical Trimming Assessment routine at step 803. This routine is valuable to the effectiveness of the compensation calculation and will now be described in detail.

FIG. 14 shows a flow chart of steps in the Historical Trimming Assessment routine. After initialisation in step 1300 upon being called from step 803 in the Test Quality Trend routine, a next step 1301 is a test to ensure that there is more than 15 minutes of data stored in the historical array. This is the same condition as will have been previously tested in the main loop at step 610. The routine should therefore not be able to initialise with a historical buffer less than 15 minutes, because the 15 minute test in step 610 will already have been failed, but once inside the routine this condition can occur following actions within the routine so a re-test for the same condition is included. As before, a different length of time can be used if preferred. If test in step 1301 shows less than 15 minutes in the array then the routine moves to step 1302 where the Stable_Flag is set to 0 to indicate that it is unlikely there is sufficient data present in the array to get a stable correction result. From there the function exits at step 1303, to return to the Test Quality Trend routine which advances to step 806. However, if there is more than the minimum time, in this case 15 minutes, of data in the historical array then the routine moves to step 1304 where a time frame is created that is shorter than the full historical array and excludes the oldest data. This time frame is designated as a short array. Step 1304 may use a fixed shortening of the data, for example, removing the oldest 5 minutes so that the short array comprises the historical array minus the oldest 5 minutes of data. Longer or shorter durations of old data might be excluded instead. Alternatively a reactive approach may be used; FIG. 13 includes an example. Under this reactive approach, the duration of the short array is calculated as the total time of the historical array multiplied by the square root of the R² value multiplied by 0.999. The square root of the R² value is used in this example as this prevents the function from being too aggressive with low values, but any function of this type will work equally well. The R² value is multiplied by 0.999 to ensure that the unlikely event of R² being 1 does not cause a program error (no reduction of the array duration). The aim of this function is to remove more old data the poorer the correlation between the sensor data Sen1 and Sen2. Poor correlation gives a low R² value, giving a smaller multiplier in the step 1304 calculation, leading to a smaller fraction of the historical array being taken as the short array. Now, the short array comprises that duration of the most recent data from the historical array. In summary, the short array may comprise the historical array minus a fixed duration of the oldest data (non-reactive approach), or a fraction of the newest data where the length of the fraction increases with R². In either case the short array is a most recent section of the full length historical array.

Moving to step 1305, the Sen1′ and Sen2′ data in the short array are taken and a linear regression is carried out to fit the short array Sen1′ against the short array Sen2′ data, as was previously done in step 608 of the main loop for the Sen1′ and Sen2′ data over the full array length. Also as before, values of R², Average Error and CtN are calculated for this model, where the underline distinguishes these statistical coefficients from those for the full length data array. At this point a counter Trim_Score is set to 0. Moving to step 1306, the two sets of statistical coefficients, those describing the historical array and those describing the short array, are compared. In this example, the coefficients compared are R², the Average Error and CtN. For each case where the R², average Error and CtN have improved for the shorter history compared with the full array, then 1 is added to the Trim_Score. For improvement, R² and CtN must increase and Average Error must decrease. So, for each of R²>R², Average Error<Average Error and CtN>CtN, the Trim_Score is increased by 1. In the next step 1307, the final Trim_Score is examined. If it is found to be less than 2 (i.e. 1 or 0), then no changes are made to the historical array size, and the routine is exited at step 1303. If the Trim_Score is 2 or more, then the values of R², Average Error and CtN for the short array are used in step 1308 to replace the stored values of R², Average Error and CtN calculated for the full historical array. Also in the next step 1309, the oldest data in the historical array which are not within the short array time frame are deleted from the historical array. In other words, the historical array is trimmed so as to exclude older data and include newer data having statistics showing better correlation between Sen1 and Sen2. This process enables the historical buffer to delete old data that is no longer constructive to the calculation of the correction factor. Then, the routine returns back to the start of the loop at step 1301, where the test for a minimum buffer length of 15 minutes is repeated followed by the rest of the routine. Consequently, the array will continue to be trimmed if continued improvement in correlation (assessed on the statistical comparisons) is found over increasingly recent fractions of data until there is no further improvement (Trim_Score less than 2). At this point, the historical array has been trimmed such that on average the test parameters are at a local maximum. The routine then exits at step 1303 to return to the main loop, where the data of the trimmed array is tested for stability in step 613, and if found sufficiently stable, is passed for calculation of the correction factor.

This trimming routine is valuable, because the assumptions made at the start of the method for the removal of the independent long term drift of the sensors, embodied in the mathematical fit modelling and error calculations of steps 604 and 606, are typically only valid over finite durations. How long the correlation between the two measured signals holds valid will vary throughout a process because this is linked to the changing of the media composition (which will not remain constant) and any other sources of drift (such as water absorption in the sensors). Without an ability to continually evaluate and adjust the duration of the historical array the calculated correlation may become dominated by the composition change (or other external factor causing drift) and not the thermal change (or noise source of interest in the given system), resulting in a correction factor which is mathematically sound but grossly incorrect. Use of such a corrected signal in subsequent operations, such as control of feeding events, will therefore produce errors. Trimming the historical array according to the routine of FIG. 14 can avoid problems of this type.

Note that any relevant statistical test could be applied to determine an improvement in the correlation fit, not just the ones discussed in the comparison tests of step 1306. As an example these could include the standard deviation (SD) with more emphasis on outliers or median average error (MAD) to reduce the dependence on outliers. Overall, the aim is to calculate one or more statistical coefficients or quantifiers of the mathematical models of the correlation between the signal data and the noise data, and compare the quantifiers to assess whether the modelling is more accurate for the recent data compared to the full data set. If the comparison shows an improvement in accuracy, the older data is removed from the data array, and the updated shortened array is used for the compensation of the signal data.

According to the description thus far, the Historical Trimming Assessment routine is called from within the Test Quality Trend routine. This prevents the trimming routine from being executed in every pass of the main loop, or when the data is deemed stable so that trimming is not necessary or advantageous. Note, however, that the trimming routine might alternatively be included at other points in the main loop, either called directed from the main loop or from within other of the described routines. It might, for example, be placed to execute immediately before the correction factor calculation, or to be called from within the correction factor calculation routine before the correction factor is calculated.

Several of the various routines described with reference to the main loop in the FIG. 5 embodiment can be omitted or modified if desired, for example to reduce the amount of processing. The various mathematical fitting and modelling procedures in steps 604, 606 and 608 can be replaced with alternative models. The selection of statistical coefficients calculated in step 602 can be reduced, increased or replaced with other statistical quantifiers, so long as the coefficients chosen are able to be used in the desired assessment of the data and its models for stability and fitting quality. The rates of change and acceleration calculated in step 609 might be replaced or supplemented with other higher order derivatives. The Test Quality Trend routine in step 611 might be omitted, or executed with tests based on alternative noise indicators and derivative values able to give an indication of the overall trend of the data and models. The Test Stability routine in step 613 can include other or different statistical comparisons to check the stability of the evolving accrued data, or might be omitted. The Historical Averaging routine in step 615 is optional or might use different techniques to reduce the overall amount of data in the array while preserving its length. The Calculate Correction Factor routine in step 617 might have other or different scaling steps applied to modify the initial correction factor. The resulting corrected parameter data might be stored for later use or analysis, and/or might be used immediately for system control. The measured parameter and its perturbing noise might be refractive index and temperature, but other parameters and noise sources may equally effectively be handled by embodiments of the invention.

An advantage of the invention is that the correlation-based compensation technique can be implemented without loss of information from the measured data. This is in contrast to noise removal techniques such as Fourier analysis that require averaging of data, leading inevitably to information loss.

Also, the methods of the inventions are entirely self-sufficient in that they require no assumptions to be made or known values to be input; the compensation is achieved solely from the signal and reference data.

The invention is especially valuable for processes yielding small signal values, where the effective of the perturbing factor might be much larger than the underlying variation in the signal which is actually of interest. The described correlation technique holds good in such a regime, whereas known correction approaches can be defective under these conditions.

While the invention has been described in terms of a method, embodiments are equally applicable to apparatus and systems configured to implement embodiments of the method, and to computer systems configured to execute routines to implement embodiments of the method, the incorporation of such computer systems into larger apparatus, and computer programs executable by a processor to implement the method in a system.

The systems and methods described herein are intended to provide a route to derive useful and sensitive compensated measurements in fields such as bioreactor kinetics, where the measurements are applicable to both process monitoring and process control. The invention proposes a technique which uses information between a signal channel and a reference or noise channel (both of which respond to an external variability) as a way to improve the sensitivity of an overall system for data logging or control.

The invention has been described with particular reference to a refractive index sensor and its use in a bioreactor control system, but the approach could be applied to other applications which have similar characteristics, specifically ones where at least two signal channels are recorded, one of which is a measurement of a parameter that depends on a quantity of interest (fluid refractive index in the particular examples) and at least one external factor (temperature in the examples), and where the second channel largely depends on the external factor, albeit with a possible different sensitivity and potential lag in data. Embodiments of the invention will be particularly useful in situations where the external factor causes large changes (in the bioreactor example the thermal fluctuations are large enough to cause detectable changes in the genuine refractive index signal and may well be larger than the genuine signal). It can work well for situations where sufficient oscillations (or variation) in the external factor occur to build up a reliable correlation before the nature of the signal and the factor causing that signal change over time; in a bioreactor system this will be the composition of the fluid media changing. Examples of systems where the invention might be applied include mixing systems (for example to produce media for bio-reactors), precipitation based processes, and polymer chain length control in synthesis of organic chemicals. Other systems and applications are not precluded, however.

### REFERENCES

[1] GB 1416233.3

[2] http://stratophase.com/products/

1. A method for compensating perturbed measurements, comprising:
obtaining measurements with respect to time of a signal representing a parameter modified by a perturbing influence and a signal representing the perturbing influence and storing the measurements for each time in a data array as parameter data and perturbation data respectively;
for each of the parameter data and the perturbation data:
fitting a mathematical model to the data with respect to time;
determining an error between the model and the data at each time;
storing the errors in the data array as normalised parameter data and normalised perturbation data respectively;
fitting a mathematical model to the normalised parameter data against the normalised perturbation data to obtain a correlation function representing correlation between the parameter data and the perturbation data;
using the correlation function to calculate a correction factor representing the modification of the parameter data by the perturbing influence;
using the correction factor to adjust the parameter data for the modification to obtained compensated parameter data; and
outputting the compensated parameter data.

2. A method according to claim 1, in which the correction factor is calculated by multiplying normalised perturbation data for a most recent time with a multiplier of a first degree of the correlation function mathematical model, and the compensated parameter data is obtained by subtracting the correction factor from the parameter data for the most recent time.

3. A method according to claim 1, further comprising:
calculating one or more statistical coefficients of the correlation function that indicate the goodness of fit of the correlation function mathematical model; and
using at least one of the statistical coefficients to scale a magnitude of the correction factor before using the correction factor to adjust the parameter data, such that the correction factor is reduced inversely with the goodness of fit, a better fit yielding a larger magnitude.

4. A method according to claim 1, in which the parameter data and the perturbation data are stored in the data array to create a full array and the correlation function is a full array correlation function; the method further comprising:
designating as a short array parameter data and perturbation data from the full array over a time period shorter than the time period of the full array and including the most recent data;
obtaining a correlation function representing correlation between the short array parameter data and the short array perturbation data as for the full array correlation function;
calculating one or more statistical coefficients of the short array correlation function and the full array correlation function that indicate the goodness of fit of the short array correlation function mathematical model and the full array correlation function mathematical model;
comparing the statisitical coefficients from the short array with the statistical coefficients from the full array to determine if the mathematical model fitting is improved for the short array compared to the full array;
updating the full array by removing parameter data and perturbation data older than the oldest time in the short array if the comparison shows an improved fit, and by retaining all the full array data otherwise; and
using the correlation function of the updated full array to calculate the correction factor.

5. A method according to claim 4, further comprising:
calculating one or more statistical coefficients of the correlation function that indicate the goodness of fit of the correlation function mathematical model; and
using at least one of the statistical coefficients to scale a magnitude of the correction factor before using the correction factor to adjust the parameter data, such that the correction factor is reduced inversely with the goodness of fit, a better fit yielding a larger magnitude and in which the statistical coefficients used to scale the magnitude of the correction factor are statistical coefficients of the updated full array.

6. A method according to claim 4, in which the time period for the short array is a fraction of the time period of the full array determined in proportion to the goodness of fit of the full array correlation function mathematical model, such that a better fit yields a larger fraction and hence a longer time period.

7. A method according to claim 1, further comprising:
calculating one or more statistical coefficients of the correlation function that indicate the goodness of fit of the correlation function mathematical model;
determining rates of change of the one or more statistical coefficients over the time of the data array;
analysing the rates of change against one or more stability criteria to assess stability of the data over time; and
if the stability criteria are met, calculating and using the correction factor, and otherwise obtaining more data until the stability criteria are met.

8. A method according to claim 1, in which the measurements are obtained from media in a bioreactor during a bioreaction.

9. A method according to claim 1, in which the parameter is refractive index and the perturbing influence is temperature.

10. A system comprising:
a first sensor configured to collect data with respect to time of a signal representing a parameter modified by a perturbing influence;
a second sensor configured to collect data with respect to time of a signal representing the perturbing influence;
memory for storing the collected data in a data array; and
a processor having program instructions configured to cause the processor to implement a method according to claim 1.

11. A system according to claim 10, in which the first sensor is a refractive index sensor and the second sensor is a temperature sensor.

12. A system according to claim 10, further comprising a bioreactor vessel, the first sensor and the second sensor arranged to collect data from media in the vessel.

13. A system according to claim 10, in which the processor is further configured to use the compensated parameter data to generate one or more control signals to control operation of one or more components of the system.

14. A system according to claim 13, in which the one or more control signals comprise signals to control delivery of additive into the bioreactor vessel.

15. A computer program product comprising a computer readable storage medium bearing instructions executable by a processor to implement a method according to claim 1.
